(19) United States
(12) Patent Application Publication
(10) Pub. No.: US 2025/0152797 A1
(43) Pub. Date: May 15, 2025

(54) APPARATUS FOR THE EXTRACORPOREAL TREATMENT OF BLOOD

(71) Applicant: University of Limerick, Limerick (IE)

(72) Inventors: Jakki COONEY, Mallow (IE); Todd Fumio KAGAWA, Kallua, HI (US); Edmond MAGNER, Drombanna (IE)

(21) Appl. No.: 18/676,785

(22) Filed: May 29, 2024

Related U.S. Application Data
(63) Continuation of application No. 17/222,278, filed on Apr. 5, 2021, now abandoned.
Application No. 17/222,278 is a continuation of application No. 16/734,733, filed on Jan. 6, 2020, now abandoned.
Application No. 16/734,733 is a continuation of application No. 15/242,775, filed on Aug. 22, 2016, now abandoned.
Application No. 15/242,775 is a continuation of application No. 14/314,387, filed on Jun. 25, 2014, now Pat. No. 9,422,541.

(30) Foreign Application Priority Data
Dec. 17, 2013 (EP) 13197790.2

(51) Int. Cl. A61M 1/36; A61L 33/00; A61M 1/34; C12N 9/52; C12N 11/14

(52) U.S. Cl. CPC A61M 1/3687; A61L 33/0047; A61M 1/3496; A61M 1/3689; C12N 9/52; C12N 11/14; C12Y 304/2111

(57) ABSTRACT

An apparatus for the extracorporeal treatment of blood comprising an extracorporeal blood circuit (2), a pump (6) configured to provide fluid displacement within the extracorporeal blood circuit, and a reaction chamber (8) connected to the extracorporeal blood circuit and configured to receive blood or plasma from the circuit and treat the blood or plasma. The reaction chamber comprises a protease enzyme immobilized to a support, in which the protease enzyme is specific for, and capable of irreversibly cleaving, a human C5a present in the blood or plasma, wherein the abundance of the human C5a in the treated blood or plasma is less than that in the untreated blood or plasma. The apparatus finds utility in the extracorporeal treatment of blood from patients with inflammatory conditions, especially auto-immune disease and sepsis.

### CROSS-REFERENCE TO RELATED APPLICATIONS

This application is a continuation of U.S. application Ser. No. 17/222,278, filed on Apr. 5, 2021, which is a continuation of U.S. application Ser. No. 16/734,733, filed on Jan. 6, 2020, which is a continuation of U.S. application Ser. No. 15/242,775 filed on Aug. 22, 2016, which is a continuation of U.S. application Ser. No. 14/314,387 filed on Jun. 25, 2014, now U.S. Pat. No. 9,422,541, which claims priority from European Patent Application No. 13197790.2, filed on Dec. 17, 2013. The contents of these previously filed applications are incorporated herein by reference in their entirety.

### REFERENCE TO AN ELECTRONIC SEQUENCE LISTING

This application is being filed electronically via Patent Center and includes an electronically submitted sequence listing in .txt format. The contents of the electronic sequence listing (AMRT_028_04US_SeqList_ST26.xml; Size: 15,460 bytes; and Date of Creation: May 28, 2024) are herein incorporated by reference in its entirety.

### BACKGROUND

State-of-the-art hospital treatment for sepsis is the implementation of ‘The Sepsis Six’ (PMID 21398303). These are a series of interventions to stabilize the patient, including delivery of antibiotics, microbial culture, delivery of high-flow oxygen, and fluids. To date, interventions to mitigate organ damage in sepsis have failed. Treatment with Drotrecogin alfa activated, a serine protease involved in switching off coagulation, was, until very recently, the major FDA-approved intervention for treatment of human sepsis. However in 2011 FDA announcing that Eli Lilly had withdrawn Xigris (Drotrecogin alfa). On the 8 Aug. 2012, AstraZeneca announced that a Phase IIb study testing the efficacy of CytoFab™, an anti-TNFα polyclonal antibody fragment, for treatment of severe sepsis and/or septic shock, did not show any significant improvement over placebo and AZ halted any further developments.

Two additional treatments have been proposed based on a blood purification strategy with some similarity to that proposed in this document. Cytosorb's IL-8 adsorption cassette is based on a porous material that adsorbs the cytokine IL-8, but the technique is non-selective, and removes other small protein components of the blood (found on the World Wide Web at cytosorbents.com/tech.htm). The second strategy is a specific adsorption resin removing bacterial LPS from blood circulated through a cassette (found on the World Wide Web at altecomedical.com/market_product.php), and is a treatment limited to sepsis caused by Gram negative bacteria.

There is a large body of evidence establishing the role of C5a in sepsis. The Cell Envelope Protease ScpA targets the immune proinflammatory mediator C5a and specifically cleaves the mediator, rendering it inactive.

It is an object of the invention to overcome at least one of the above-referenced problems.

### SUMMARY

The invention is based on a method and device for the extracorporeal treatment of inflammatory conditions in a patient, especially auto-immune diseases, sepsis or septicemia, that involves reacting blood that has been removed from a patient with a protease enzyme immobilized to a support in which the enzyme is specific for a pro-inflammatory mediator present in the blood of the patient and is capable of cleaving the pro-inflammatory mediator and thereby reducing the abundance of pro-inflammatory mediator in the blood of the patient prior to the return of the treated blood to the patient.

In a first aspect, the invention relates to an apparatus for the extracorporeal treatment of blood comprising:
an extracorporeal blood circuit;
optionally, a pump configured to provide fluid displacement within the extracorporeal blood circuit; and
a reaction chamber connected to the extracorporeal blood circuit and configured to receive blood or a pro-inflammatory mediator containing blood fraction from the circuit and treat the blood or pro-inflammatory mediator containing blood fraction, characterized in that the reaction chamber comprises a protease enzyme irreversibly immobilized to a support, in which the protease enzyme is specific for, and capable of irreversibly cleaving, a human pro-inflammatory mediator present in the blood or plasma such that the chemoattractant capability of the pro-inflammatory mediator is reduced or preferably abrogated, wherein the abundance of functional pro-inflammatory mediator in the treated blood or plasma is less than that in the untreated blood or plasma.

Compared with extracorporeal treatment devices that operate on the basis of adsorption of pro-inflammatory mediators, the apparatus of the invention has a number of advantages. Each molecule of enzyme can cleave a large number of molecules of substrate during a treatment operation; this contrasts with the adsorption process in which the ligand, once bound to its target molecule, is unavailable for binding with further target molecules. Second, the affinity antibody-based approaches of the prior art are susceptible to cross-reacting with non-target molecules, and involve significant costs in the development and generation of suitable antibodies. In contracts, enzymes that are specific to pro-inflammatory mediators are known from the literature, and can be easily produced using recombinant DNA technology.

Preferably, the pro-inflammatory mediator is selected from a group consisting of, but not limited to: C3a, C4a, C5a, IL-8, IL-6, TNFα, IL-1, or Mig. Thus, in one embodiment, the protease enzyme is capable of cleaving a human pro-inflammatory mediator selected from a group consisting of, but not limited to, C3a, C4a, C5a, IL-8, IL-6, TNFα, IL-1, and Mig.

In a preferred embodiment, the invention provides an apparatus for the extracorporeal treatment of blood comprising:
an extracorporeal blood circuit;
optionally, a pump configured to provide fluid displacement within the extracorporeal blood circuit; and
a reaction chamber connected to the extracorporeal blood circuit and configured to receive blood or a human C5a-containing blood fraction from the circuit and treat the blood or human C5a-containing blood fraction,

characterized in that the reaction chamber comprises a protease enzyme irreversibly immobilized to a support, in which the protease enzyme is specific for, and capable of irreversibly cleaving, human C5a present in the blood or blood fraction such that the chemoattractant capability of the cleaved human C5a is reduced, wherein the abundance of the functional human C5a in the treated blood or blood fraction is less than that in the untreated blood or blood fraction.

As used herein, the term “functional human C5a” should be understood to mean human C5a having chemoattractant capability as determined using the chemoattractant capability assay described below. Likewise, the term “non-functional human C5a” should be understood to mean cleaved C5a protein that has reduced, or is devoid of, chemoattractant capability as determined using the chemoattractant capability assay described below.

The invention also provides an apparatus for treating human blood or a pro-inflammatory mediator-containing blood fraction, the apparatus comprising a protease enzyme irreversibly bound to a support, in which the protease enzyme is specific for, and capable of irreversibly cleaving, a pro-inflammatory mediator present in the blood or blood fraction such that the chemoattractant capability of the cleaved human pro-inflammatory mediator is reduced.

The invention also provides an apparatus for treating human blood or a C5a-containing blood fraction, the apparatus comprising a protease enzyme irreversibly bound to a support, in which the protease enzyme is specific for, and capable of irreversibly cleaving, human C5a present in the blood or blood fraction such that the chemoattractant capability of the cleaved human C5a is reduced.

The invention also provides a protease enzyme comprising the sequence of A-B-C-D, in which:
A is a protease enzyme that is specific for, and capable of irreversibly cleaving, a human pro-inflammatory mediator present in the blood such that the chemoattractant capability of the cleaved pro-inflammatory mediator is reduced, B is a poly-lysine, poly-cysteine or poly-glutamate motif, C is a spacer (for example a short peptide of 2 to 20 amino acids), and D is a poly-histidine motif.

Preferably, the protease enzyme is a recombinant bacterial C5a protease comprising a sequence of SEQ ID NO: 3 or a functional variant thereof, typically having at least 70%, 80% or 90% sequence identity with SEQ ID NO: 3.

The term “functional variant” as applied to SEQ ID NO: 3 means a protease that is specific for, and capable of irreversibly cleaving, human C5a such that the chemoattractant capability of the cleaved human C5a is reduced, or preferably abrogated.

Examples functional variants of SEQ ID NO: 3 are selected from SEQ ID NO: 4 and SEQ ID NO: 5.

In one embodiment, the apparatus of the invention includes separating means adapted to separate the blood into a C5a-containing fraction and a non-C5a containing fraction, wherein the reaction chamber receives the C5a-containing fraction. The separating means could be, for example, a filter configured to separate the blood or a fraction thereof into a low-molecular weight containing fraction and a second fraction, wherein the low molecular weight containing fraction is the C5a containing fraction.

Suitably, the apparatus of the invention includes means configured to recombine the treated C5a-containing fraction (i.e. the low molecular weight fraction) with the second non-C5a containing fraction. The recombined fractions are then returned to the patient.

In one preferred embodiment of the invention, a C-terminal of the protease enzyme comprises a first tag and a second tag located distally of the first tag and separated from the first tag by a spacer. Typically, the support comprises a coordinated transition metal ion and one or more functional groups. Suitably, the first tag comprises a motif capable of covalently reacting with the one or more functional groups, and wherein the second tag comprises a motif capable of interacting with the coordinated transition metal ion.

In this manner, the protease enzyme can be oriented with respect to the surface such that the C-terminus of the enzyme is disposed adjacent to the surface (this is achieved by the interaction between the second tag and the coordinated transition metal of the support surface), thus allowing the adjacent first tag to covalently bind to the functional groups on the surface. This will prevent unspecific binding between functional groups on the surface and lysine residues in the protease enzyme.

Preferably, the first tag is selected from poly-lysine, poly-glutamate, or poly-cysteine tag, and the functional groups on the surface are groups that are capable of covalently binding with these motifs.

Suitably, the second tag comprises a poly-histidine tag or another tag capable of interaction with a transition metal.

Preferably, the coordinated transition metal ion is selected from Ni²⁺, Co²⁺, Zn²⁺, and Cu²⁺.

Both tags can be appended onto the DNA sequence by PCR based methods using an oligonucleotide synthesized to contain the required sequence.

Typically, the support comprises a silica material, preferably a mesoporous silica material, preferably modified monodispersed mesoporous silicate material, and ideally a Ni²⁺-modified mesoporous silica material. Other potential materials for the support include but are not limited to methacrylates, polyacrylamides, polypyrroles and polysaccharides.

Suitably, the support comprises a bead. Preferably, the reaction chamber comprises a column containing a multiplicity of beads.

The invention also relates to an apparatus of the invention for use in a method for the ex-vivo treatment of blood in a mammal, typically a human. Preferably, the mammal has an inflammatory condition such as sepsis.

The nucleic acid sequence encoding the bacterial C5a pro-protease, ScpA from Streptococcus pyogenes, is provided in SEQ ID NO: 1 below:

DNA sequence
(SEQ ID NO: 1)
GGATCCAATACTGTGACAGAAGACACTCCTGCTACCGAACAAGCCGTAGA
AACCCCACAACCAACAGCGGTTTCTGAGGAAGCACCATCATCATCAAAGG
AAACCAAAATCCCACAAACTCCTGGTGATGCAGAAGAAACAGTAGCAGAT
GACGCTAATGATCTAGCCCCTCAAGCTCCTGCTAAAACTGCTGATACACC
AGCAACCTCAAAAGCGACTATTAGGGATTTGAACGACCCTTCTCAGGTCA
AAACCCTGCAGGAAAAAGCAGGCAAGGGAGCTGGGACTGTTGTTGCAGTG
ATTGATGCTGGTTTTGATAAAAATCATGAAGCGTGGCGCTTAACAGACAA
AACTAAAGCACGTTACCAATCAAAAGAAGATCTTGAAAAAGCTAAAAAAG
AGCACGGTATTACCTATGGCGAGTGGGTCAATGATAAGGTTGCTTATTAC
CACGATTATAGTAAAGATGGTAAAACCGCTGTCGATCAAGAGCACGGCAC
ACACGTGTCAGGGATCTTGTCAGGAAATGCTCCATCTGAAACGAAAGAAC
CTTACCGCCTAGAAGGTGCGATGCCTGAGGCTCAATTGCTTTTGATGCGT
GTCGAAATTGTAAATGGACTAGCAGACTATGCTCGTAACTACGCTCAAGC
TATCAGAGATGCTGTCAACTTGGGAGCTAAGGTGATTAATATGAGCTTTG
GTAATGCTGCACTAGCTTACGCCAACCTTCCAGACGAAACCAAAAAAGCC
TTTGACTATGCCAAATCAAAAGGTGTTAGCATTGTGACCTCAGCTGGTAA
TGATAGTAGCTTTGGGGGCAAAACCCGTCTACCTCTAGCAGATCATCCTG
ATTATGGGGTGGTTGGGACGCCTGCAGCGGCAGACTCAACATTGACAGTT
GCTTCTTACAGCCCAGATAAACAGCTCACTGAAACTGCTACGGTCAAAAC
AGACGATCATCAAGCTAAAGAAATGCCTGTTCTTTCAACAAACCGTTTTG
AGCCAAACAAGGCTTACGACTATGCTTATGCTAATCGTGGGATGAAAGAA
GATGATTTTAAGGATGTCAAAGGCAAAATTGCCCTTATTGAACGTGGTGA
TATTGATTTCAAAGATAAGATTGCAAACGCTAAAAAAGCTGGTGCTGTAG
GGGTCTTGATCTATGACAATCAAGACAAGGGCTTCCCGATTGAATTGCCA
AATGTTGATCAGATGCCTGCGGCCTTTATCAGTCGAAAAGACGGTCTCTT
ATTAAAAGACAATTCTAAAAAAACCATCACCTTCAATGCGACACCTAAGG
TATTGCCAACAGCAAGTGACACCAAACTAAGCCGCTTCTCAAGCTGGGGT
TTGACAGCTGACGGCAATATTAAGCCAGATATTGCAGCACCCGGCCAAGA
TATTTTGTCATCAGTGGCTAACAACAAGTATGCCAAACTTTCTGGAACTA
GTATGTCTGCGCCATTGGTAGCGGGTATCATGGGACTATTGCAAAAGCAA
TATGAGACACAGTATCCTGATATGACACCATCAGAGCGTCTTGATTTAGC
TAAAAAAGTATTGATGAGCTCAGCAACTGCCTTATATGATGAAGATGAAA
AAGCTTATTTTTCTCCTCGCCAACAAGGAGCAGGAGCAGTCGATGCTAAA
AAAGCTTCAGCAGCAACGATGTATGTGACAGATAAGGACAATACCTCAAG
CAAGGTTCACCTGAACAATGTTTCTGATAAATTTGAAGTAACAGTAACAG
TTCACAACAAATCTGATAAACCTCAAGAGTTGTATTACCAAGCAACTGTT
CAAACAGATAAAGTAGATGGAAAACACTTTGCCTTGGCTCCTAAAGCATT
GTATGAGACATCATGGCAAAAAATCACAATTCCAGCCAATAGCAGCAAAC
AAGTCACCGTTCCAATCGATGCTAGTCGATTTAGCAAGGACTTGCTTGCC
CAAATGAAAAATGGCTATTTCTTAGAAGGTTTTGTTCGTTTCAAACAAGA
TCCTAAAAAAGAAGAGCTTATGAGCATTCCATATATTGGTTTCCGAGGTG
ATTTTGGCAATCTGTCAGCCTTAGAAAAACCAATCTATGATAGCAAAGAC
GGTAGCAGCTACTATCATGAAGCAAATAGTGATGCCAAAGACCAATTAGA
TGGTGATGGATTACAGTTTTACGCTCTGAAAAATAACTTTACAGCACTTA
CCACAGAGTCTAACCCATGGACGATTATTAAAGCTGTCAAAGAAGGGGTT
GAAAACATAGAGGATATCGAATCTTCAGAGATCACAGAAACCATTTTTGC
AGGTACTTTTGCAAAACAAGACGATGATAGCCACTACTATATCCACCGTC
ACGCTAATGGCAAACCATATGCTGCGATCTCTCCAAATGGGGACGGTAAC
AGAGATTATGTCCAATTCCAAGGTACTTTCTTGCGTAATGCTAAAAACCT
TGTGGCTGAAGTCTTGGACAAAGAAGGAAATGTTGTTTGGACAAGTGAGG
TAACCGAGCAAGTTGTTAAAAACTACAACAATGACTTGGCAAGCACACTT
GGTTCAACCCGTTTTGAAAAAACGCGTTGGGACGGTAAAGATAAAGACGG
CAAAGTTGTTGTTAACGGAACCTACACCTATCGTGTCCGCTACACTCCGA
TTAGCTCAGGTGCAAAAGAACAACACACTGATTTTGATGTGATTGTAGAC
AATACGACACCTGAAGTCGCAACATCGGCAACATTCTCAACAGAAGATCG
TCGTTTGACACTTGCATCTAAACCAAAAACCAGCCAACCGATTTACCGTG
AGCGTATTGCTTACACTTATATGGATGAGGATCTGCCAACAACAGAGTAT
ATTTCTCCAAATGAAGATGGTACCTTTACTCTTCCTGAAGAGGCTGAAAC
AATGGAAGGCGGTACTGTTCCATTGAAAATGTCAGACTTTACTTATGTTG
TTGAAGATATGGCTGGTAACATCACTTATACACCAGTGACTAAGCTATTG
GAGGGCCACTCTTAA

The amino acid sequence of the bacterial C5a pro-protease, ScpA from Streptococcus pyogenes, is provided in SEQ ID NO: 2 below:

Protein sequence
(SEQ ID NO: 2)
GPLGSNTVTEDTPATEQAVETPQPTAVSEEAPSSSKETKIPQTPGDAEET
VADDANDLAPQAPAKTADTPATSKATIRDLNDPSQVKTLQEKASKGAGTV
VAVIDAGFDKNHEAWRLTDKTKARYQSKEDLEKAKKEHGITYGEWVNDKV
AYYHDYSKDGKTAVDQEHGTHVSGILSGNAPSETKEPYRLEGAMPEAQLL
LMRVEIVNGLADYARNYAQAIRDAVNLGAKVINMSFGNAALAYANLPDET
KKAFDYAKSKGVSIVTSAGNDSSFGGKTRLPLADHPDYGVVGTPAAADST
LTVASYSPDKQLTETATVKTDDHQAKEMPVLSTNRFEPNKAYDYAYANRG
MKEDDFKDVKGKIALIERGDIDFKDKIANAKKAGAVGVLIYDNQDKGFPI
ELPNVDQMPAAFISRKDGLLLKDNSKKTITENATPKVLPTASDTKLSRES
SWGLTADGNIKPDIAAPGQDILSSVANNKYAKLSGTSMSAPLVAGIMGLL
QKQYETQYPDMTPSERLDLAKKVLMSSATALYDEDEKAYFSPRQQGAGAV
DAKKASAATMYVTDKDNTSSKVHLNNVSDKFEVTVTVHNKSDKPQELYYQ
ATVQTDKVDGKHFALAPKALYETSWQKITIPANSSKQVTVPIDASRFSKD
LLAQMKNGYFLEGFVRFKQDPKKEELMSIPYIGFRGDFGNLSALEKPIYD
SKDGSSYYHEANSDAKDQLDGDGLQFYALKNNFTALTTESNPWTIIKAVK
EGVENIEDIESSEITETIFAGTFAKQDDDSHYYIHRHANGKPYAAISPNG
DGNRDYVQFQGTFLRNAKNLVAEVLDKEGNVVWTSEVTEQVVKNYNNDLA
STLGSTRFEKTRWDGKDKDGKVVVNGTYTYRVRYTPISSGAKEQHTDFDV
IVDNTTPEVATSATFSTEDRRLTLASKPKTSQPIYRERIAYTYMDEDLPT
TEYISPNEDGTFTLPEEAETMEGGTVPLKMSDFTYVVEDMAGNITYTPVT
KLLEGHS

The amino acid sequence of mature bacterial C5a protease, ScpA from Streptococcus pyogenes, is provided in SEQ ID NO: 3 below:

AEETVADDANDLAPQAPAKTADTPATSKATIRDLNDPSQVKTLQEKASKG
AGTVVAVIDAGFDKNHEAWRLTDKTKARYQSKEDLEKAKKEHGITYGEWV
NDKVAYYHDYSKDGKTAVDQEHGTHVSGILSGNAPSETKEPYRLEGAMPE
AQLLLMRVEIVNGLADYARNYAQAIRDAVNLGAKVINMSFGNAALAYANL
PDETKKAFDYAKSKGVSIVTSAGNDSSFGGKTRLPLADHPDYGVVGTPAA
ADSTLTVASYSPDKQLTETATVKTDDHQAKEMPVLSTNRFEPNKAYDYAY
ANRGMKEDDFKDVKGKIALIERGDIDFKDKIANAKKAGAVGVLIYDNQDK
GFPIELPNVDQMPAAFISRKDGLLLKDNSKKTITENATPKVLPTASDTKL
SRFSSWGLTADGNIKPDIAAPGQDILSSVANNKYAKLSGTSMSAPLVAGE
VIGLLQKQYETQYPDMTPSERLDLAKKVLMSSATALYDEDEKAYFSPRQQ
GAGAVDAKKASAATMYVTDKDNTSSKVHLNNVSDKFEVTVTVHNKSDKPQ
ELYYQATVQTDKVDGKHFALAPKALYETSWQKITIPANSSKQVTVPIDAS
RFSKDLLAQMKNGYFLEGFVRFKQDPKKEELMSIPYIGFRGDFGNLSALE
KPIYDSKDGSSYYHEANSDAKDQLDGDGLQFYALKNNFTALTTESNPWTI
IKAVKEGVENIEDIESSEITETIFAGTFAKQDDDSHYYIHRHANGKPYAA
ISPNGDGNRDYVQFQGTFLRNAKNLVAEVLDKEGNVVWTSEVTEQVVKNY
NNDLASTLGSTRFEKTRWDGKDKDGKVVVNGTYTYRVRYTPISSGAKEQH
TDFDVIVDNTTPEVATSATFSTEDRRLTLASKPKTSQPIYRERIAYTYMD
EDLPTTEYISPNEDGTFTLPEEAETMEGGTVPLKMSDFTYVVEDMAGNIT
YTPVTKLLEGHS

The amino acid sequence of a first variant of mature bacterial C5a protease, ScpA from Streptococcus pyogenes, is provided in SEQ ID NO: 4 below:

DANDLAPQAPAKTADTPATSKATIRDLNDPSQVKTLQEKASKGAGTVVAV
IDAGFDKNHEAWRLTDKTKARYQSKEDLEKAKKEHGITYGEWVNDKVAYY
HDYSKDGKTAVDQEHGTHVSGILSGNAPSETKEPYRLEGAMPEAQLLLMR
VEIVNGLADYARNYAQAIRDAVNLGAKVINMSFGNAALAYANLPDETKKA
FDYAKSKGVSIVTSAGNDSSFGGKTRLPLADHPDYGVVGTPAAADSTLTV
ASYSPDKQLTETATVKTDDHQAKEMPVLSTNRFEPNKAYDYAYANRGMKE
DDFKDVKGKIALIERGDIDEKDKIANAKKAGAVGVLIYDNQDKGFPIELP
NVDQMPAAFISRKDGLLLKDNSKKTITFNATPKVLPTASDTKLSRFSSWG
LTADGNIKPDIAAPGQDILSSVANNKYAKLSGTSMSAPLVAGEVIGLLQK
QYETQYPDMTPSERLDLAKKVLMSSATALYDEDEKAYFSPRQQGAGAVDA
KKASAATMYVTDKDNTSSKVHLNNVSDKFEVTVTVHNKSDKPQELYYQAT
VQTDKVDGKHFALAPKALYETSWQKITIPANSSKQVTVPIDASRFSKDLL
AQMKNGYFLEGFVRFKQDPKKEELMSIPYIGFRGDFGNLSALEKPIYDSK
DGSSYYHEANSDAKDQLDGDGLQFYALKNNFTALTTESNPWTIIKAVKEG
VENIEDIESSEITETIFAGTFAKQDDDSHYYIHRHANGKPYAAISPNGDG
NRDYVQFQGTFLRNAKNLVAEVLDKEGNVVWTSEVTEQVVKNYNNDLAST
LGSTRFEKTRWDGKDKDGKVVVNGTYTYRVRYTPISSGAKEQHTDFDVIV
DNTTPEVATSATFSTEDRRLTLASKPKTSQPIYRERIAYTYMDEDLPTTE
YISPNEDGTFTLPEEAETMEGGTVPLKMSDFTYVVEDMAGNITYTPVTKL
LEGHS

The amino acid sequence of a second variant of mature bacterial C5a protease, ScpA from Streptococcus pyogenes, is provided in SEQ ID NO: 5 below:

KTADTPATSKATIRDLNDPSQVKTLQEKASKGAGTVVAVIDAGFDKNHEA
WRLTDKTKARYQSKEDLEKAKKEHGITYGEWVNDKVAYYHDYSKDGKTAV
DQEHGTHVSGILSGNAPSETKEPYRLEGAMPEAQLLLMRVEIVNGLADYA
RNYAQAIRDAVNLGAKVINMSFGNAALAYANLPDETKKAFDYAKSKGVSI
VTSAGNDSSFGGKTRLPLADHPDYGVVGTPAAADSTLTVASYSPDKQLTE
TATVKTDDHQAKEMPVLSTNRFEPNKAYDYAYANRGMKEDDFKDVKGKIA
LIERGDIDFKDKIANAKKAGAVGVLIYDNQDKGFPIELPNVDQMPAAFIS
RKDGLLLKDNSKKTITFNATPKVLPTASDTKLSRFSSWGLTADGNIKPDI
AAPGQDILSSVANNKYAKLSGTSMSAPLVAGEVIGLLQKQYETQYPDMTP
SERLDLAKKVLMSSATALYDEDEKAYFSPRQQGAGAVDAKKASAATMYVT
DKDNTSSKVHLNNVSDKFEVTVTVHNKSDKPQELYYQATVQTDKVDGKHF
ALAPKALYETSWQKITIPANSSKQVTVPIDASRFSKDLLAQMKNGYFLEG
FVRFKQDPKKEELMSIPYIGFRGDFGNLSALEKPIYDSKDGSSYYHEANS
DAKDQLDGDGLQFYALKNNFTALTTESNPWTIIKAVKEGVENIEDIESSE
ITETIFAGTFAKQDDDSHYYIHRHANGKPYAAISPNGDGNRDYVQFQGTF
LRNAKNLVAEVLDKEGNVVWTSEVTEQVVKNYNNDLASTLGSTRFEKTRW
DGKDKDGKVVVNGTYTYRVRYTPISSGAKEQHTDFDVIVDNTTPEVATSA
TFSTEDRRLTLASKPKTSQPIYRERIAYTYMDEDLPTTEYISPNEDGTFT
LPEEAETMEGGTVPLKMSDFTYVVEDMAGNITYTPVTKLLEGHS

The proteases of SEQ ID NO:s 2, 3, 4 and 5 are all capable of cleaving human C5a such that the chemoattractant capability of the cleaved protease is abrogated.

The amino acid sequence of C5a protein is provided in SEQ ID NO: 6 below.

C5a protein
(SEQ ID NO: 6)
MLQKKIEEIAAKYKHSVVKKCCYDGACVNNDETCEQRAARISLGPRCIKA
FTECCVVASQLRANISHKDMQLGR

Other proteases that are specific to, and capable of cleaving, human C5a include ScpB from Streptococcus agalactiae, and functional variants thereof (Brown et al). Examples of protease enzymes capable of specifically cleaving IL-8 include ScpC from Streptococcus pyogenes, SpyCEP from Streptococcus agalactiae and functional variants thereof (Fritzer et al, Kaur et al, Zinkernagel et al, Sjolinder et al, and Hidalgo et al)

Examples of protease enzymes capable of specifically cleaving IL-6 include a published Pseudomonas enzyme which degrades it completely (Matheson et al). Also gingipains K and R seem to have degrading activity against several mediators, but lack specificity required.

Suitably, the apparatus further comprising means of separating whole blood into a plasma fraction and a cellular fraction, and means for recombining the cellular fraction with the treated plasma fraction. In a separation process, the plasma in the patient's blood is typically segregated from its remaining constituents. The separated plasma is mixed with an acetate buffer saturated with heparin. This lowers the plasma's degree of acidity (pH value) to 5.12, causing the LDL cholesterol, Lp(a) and fibrinogen to drop selectively out of the plasma. Together with the heparin additive, the separated constituents form insoluble precipitates which can be removed from the plasma in a single filtration stage. Unused surplus heparin is held back in a separate adsorber, and bicarbonate ultrafiltration is used to restore the purified plasma to the physiologically acceptable level. The selectively treated, purified plasma is then remixed with the remaining blood constituents and supplied back to the patient. During H.E.L.P. apheresis, these four steps (plasma separation, precipitation with subsequent filtration, heparin adsorption and ultrafiltration) are performed by a single device, the PLASMAT Futura. Examples of devices capable of separating whole blood into a plasma fraction and a cellular fraction in extracorporeal blood circuits are known to the person skilled in the art, and include plasmaphoresis equipment (for example B Braun PLASMAT Futura) and hemodialysis equipment (for example Gambro PHEONIX found on the World Wide Web at gambro.com/en/global/Products/Hemodialysis/Monitors/Phoenix-dialysis-system/).

Typically, the reaction chamber comprises a column comprising beads in which the enzyme is immobilized to the beads. Alternatively, the reaction chamber may comprise a cartridge.

In a further aspect, the invention relates to a method for the treatment or prevention of an inflammatory condition in a human comprising the steps of reacting blood that has been removed from the patient, or a pro-inflammatory mediator containing fraction of the blood, with a protease enzyme immobilized to a support, in which the protease enzyme is specific for, and capable of irreversibly cleaving, a human pro-inflammatory mediator present in the blood or fraction such that the chemoattractant capability of the pro-inflammatory mediator is reduced or preferably abrogated, wherein the abundance of functional pro-inflammatory mediator in the treated blood or fraction is less than that in the untreated blood or fraction.

Typically, the human pro-inflammatory mediator is selected from the group consisting of, but not limited to, C3a, C4a, C5a, IL-8, IL-6, TNFα, IL-1, and Mig.

In a further aspect, the invention relates to a method for the treatment or prevention of an inflammatory condition in a human comprising the steps of reacting blood that has been removed from the patient, or a pro-inflammatory mediator containing fraction of the blood, with a protease enzyme immobilized to a support, in which the protease enzyme is specific for, and capable of irreversibly cleaving, human C5a present in the blood or fraction such that the chemoattractant capability of the cleaved human C5a is reduced or preferably abrogated, wherein the abundance of functional C5a in the treated blood or fraction is less than that in the untreated blood or plasma.

Suitably, the method includes the steps of separating the blood into a plasma fraction and a cellular fraction, treating the plasma fraction, and then recombining the cellular fraction with the treated plasma fraction prior to returning the blood to the patient.

Alternatively, or in addition, the method includes the steps of separating the blood into a C5a containing fraction (for example, a low molecular weight fraction) fraction and a second fraction, treating the C5a containing fraction, and then recombining the second fraction with the treated C5a containing fraction prior to returning the blood to the patient.

Typically, the method is carried out in a continuous fashion using an extracorporeal blood circuit.

Suitably, the protease enzyme is a recombinant protein.

The invention also relates to a support and a recombinant protease enzyme immobilized to the support, in which the recombinant protease enzyme comprises a C-terminal poly-histidine tag and a C-terminal poly-lysine tag, and in which the recombinant protease enzyme comprises a protease that is specific for, and capable of irreversibly cleaving, a human pro-inflammatory mediator present in the blood or plasma.

In this specification, the term “extracorporeal blood circuit” should be understood to mean an arrangement of conduits capable of removing blood from the body for treatment outside of the body and returning the thus treated blood to the body.

In this specification, the term “reaction chamber” should be understood to mean a chamber adapted to receive blood or plasma from the extracorporeal blood circuit and allow contact between the blood or plasma and protease enzyme that is immobilized to a support within the reaction chamber.

In this specification, the term “plasma” should be understood to mean blood from which cells have been fully or partially removed.

In this specification, the term “pro-inflammatory mediator” should be understood to mean a host proteinaceous entity produced in the auto-immune or sepsis response which stimulates other components of the host immune system, in particular causing migration or stimulation of leukocytes of any class and progenitor forms of these cells. Specific examples of pro-inflammatory mediators specific to the human inflammatory response include C3a, C4a, C5a, IL-8, IL-6, TNFα, IL-1, and Mig.

In the specification, the term “protease enzyme that is specific for a human pro-inflammatory mediator” should be understood to mean an enzyme with the capacity to selectively, or ideally solely, break peptide bonds of pro-inflammatory mediators of human origin by hydrolysis. The protease may also be derived from the parent protease, and modified to include a functionalization group, for example one or more of a poly-histidine, poly-lysine, or poly-glutamic acid tag.

In this specification, the term “functional variant thereof” as applied to a specific protease enzyme should be understood to mean a variant of the protease enzyme that retains the ability to specifically bind and irreversibly cleave the target pro-inflammatory mediator such that the chemoattractant activity of the cleaved pro-inflammatory mediator is reduced or abrogated. Thus, for example, a functional variant of ScpA from Streptococcus pyogenes includes variant ScpA proteases that have the ability to specifically bind and irreversibly cleave the human C5a protein such that the chemoattractant capability of the cleaved protease is reduced or abrogated, and include ScpA proteases from Streptococcus pyogenes (SEQ ID NO:2, 3, 4, 5) and other Streptococcal species. The term “variant” should be understood to mean proteins or polypeptides that have at least 70% sequence homology with the reference protease, and that are altered in respect of one or more amino acid residues. Preferably such alterations involve the insertion, addition, deletion and/or substitution of 20, 10, 5 or fewer amino acids, more preferably of 4 or fewer, even more preferably of 3 or fewer, most preferably of 1 or 2 amino acids only. Insertion, addition, and substitution with natural and modified amino acids is envisaged. The variant may have conservative amino acid changes, wherein the amino acid being introduced is similar structurally, chemically, or functionally to that being substituted. Typically, proteins which have been altered by substitution or deletion of catalytically-important residues will be excluded from the term “variant”. For any given protease enzyme, details of such catalytically-important residues will be well known to those skilled in the art. Generally, the variant will have at least 70% amino acid sequence homology, preferably at least 80% sequence homology, more preferably at least 90% sequence homology, and ideally at least 95%, 96%, 97%, 98% or 99% sequence homology with the reference protease. In this context, sequence homology comprises both sequence identity and similarity, i.e. a polypeptide sequence that shares 90% amino acid homology with wild-type bacterial mature C5a peptidase is one in which any 90% of aligned residues are either identical to, or conservative substitutions of, the corresponding residues in wild-type bacterial C5a peptidase. Substitution may be conservative or non-conservative substitution, and may involve use of natural amino acids or amino acid analogues.

The term “variant” is also intended to include chemical derivatives of a protease, i.e. where one or more residues of a protease is chemically derivatized by reaction of a functional side group. Also included within the term variant are protease molecules in which naturally occurring amino acid residues are replaced with amino acid analogues.

Proteins and polypeptides (including variants and fragments thereof) of and for use in the invention may be generated wholly or partly by chemical synthesis or by expression from nucleic acid. The proteins and peptides of and for use in the present invention can be readily prepared according to well-established, standard liquid or, preferably, solid-phase peptide synthesis methods known in the art (see, for example, J. M. Stewart et al).

In this specification, the term “inflammatory condition” means a condition in which the host mounts a response to an assault. Examples of imflammatory conditions include chronic or acute inflammatory conditions including sepsis, septic shock, systemic inflammatory response syndrome, multiple organ dysfunction syndrome, hyper-reactive airway disease, allergic reaction.

In a different aspect, the invention provides a method of attaching a molecule comprising a polyaminoacid sequence to a surface, in which the C-terminal of the protease enzyme comprises a first tag and a second tag located distally of the first tag and separated from the first tag by a spacer, and in which the support comprises a coordinated transition metal ion and one or more functional groups, and in which the first tag comprises a motif capable of covalently reacting with the one or functional groups, and wherein the second tag comprises a motif capable of interacting with the coordinated transition metal ion, the method comprising the step of reacting the molecule comprising a polyaminoacid sequence with the surface.

### BRIEF DESCRIPTION OF THE DRAWINGS

The invention description below refers to the accompanying drawings, of which:

FIG. 1 is a Diagrammatic representation of blood purifying invention.

The diagram shows the components and blood flow route envisaged for the implementation of the invention. Blood is removed from the patient and fractionated into a high protein plasma fraction and a high blood cell fraction. The former is passed over the active material (immobilized enzyme) in the reaction chamber and then recombined with the latter before return to the patient. Components of the invention are labeled: 1 the overall invention, 2 the extracorporeal blood purification device, 3 blood withdrawal line, 4 patient arm, 5 blood return line, 6 pumping system, 7 blood separator, 8 reaction chamber, 9 cartridge housing blood separation chambers, 10a and 10b blood separation chambers, 11 biocompatible size restrictive semi-permeable membrane, 12 line delivering protein rich plasma to reaction chamber, 13 line delivering blood cell rich fraction to mixing chamber, 14 line delivering treated plasma to mixing chamber, 15 mixing chamber for blood reconstitution, 16 vessel housing active component of reaction chamber, 17 reactive material comprising immobilized enzyme irreversibly coupled to solid support material.

FIG. 2a shows SDS-PAGE analysis of C5a untreated (−) and treated (+) with ScpA; and

FIG. 2b shows the scissile bond in the C5a sequence confirmed by Mass Spec analysis of C5a cleaved with ScpA.

### DETAILED DESCRIPTION

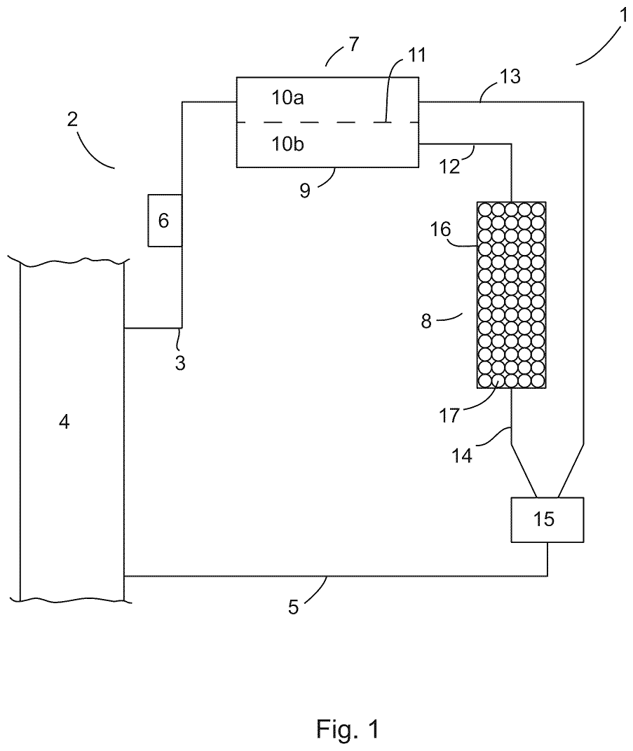

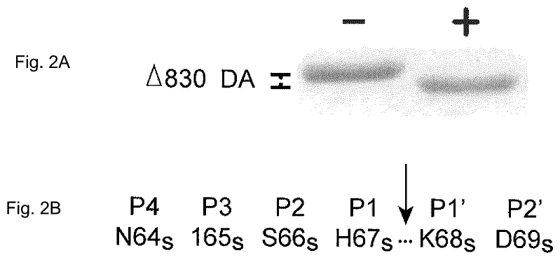

Referring to FIG. 1, there is provided an apparatus for the extracorporeal treatment of blood according to the invention, and indicated generally by the reference numeral 1. The apparatus 1 comprises an extracorporeal blood circuit 2, having a feed line 3 for withdrawing blood from a patients arm 4 for treatment and a return line 5 for returning treated blood to the patient, and an adjustable pump 6 provided in the feed line for providing blood displacement within the blood circuit 2.

The apparatus also includes a blood separator 7 and a reaction chamber 8 in the circuit 2, the separator 7 being provided upstream of the reaction chamber 8. The separator comprises a cartridge 9 having two chambers 10a and 10b separated by a semi-permeable membrane 11 adapted to allow separation of blood proteins from blood cells. The whole blood passes from the patient to the first chamber 10a, where proteins in blood plasma pass into the second chamber 10b forming a protein rich plasma fraction in the second chamber and leaving blood cells in the first chamber 10a. A tube 12 is provided to transfer the thus-formed protein rich fraction plasma from the second chamber 10b to the reaction chamber 8 where it is treated. A further tube 13 is provided to transfer the cell rich fraction from the first chamber 10a to re-join with treated plasma distally of the reaction chamber 8 at a mixing chamber 15 where the two fractions are mixed prior to being returned to the patient via the whole blood return line 5.

The reaction chamber comprises a cylindrical vessel 16 filled with functionalized support material 17 containing the immobilized enzyme, thereby providing a large surface area for the treatment of the incoming plasma. The tube 12 feeds into a top of the cylindrical vessel 16, and the plasma filters through the cylinder before exiting the vessel through a tube 14.

Mesoporous silica (MPS) materials (including but not limited to MCM, SBA, MCF and PMO type materials) are prepared using a templated synthesis method. Ideally these particles will be monodispersed in nature. The particles will have a specific particle size in the range of 0.1-50 μm, contain nanopores with a final internal diameter in the range 8-12 nm and have a high surface area 300-800 m²g⁻¹.

The surface characteristics of the silica nanocarriers will be modified with a range of functional groups (e.g. —NH2, —COOH, —SH) directly during synthesis of the material, or by post synthesis grafting to facilitate covalent coupling (through the poly-Gluamate or poly-Lysine or Cysteine residues respectively) of the enzyme to the surface after orientation specific adsorption.

The Ni²⁺-modified MPS will be prepared by attachment of 3-iodo-trimethoxypropylsilane to the silicate surface followed by reaction with cyclam and incorporation of the metal ion. This is to generate immobilization of the protease in a controlled orientation.

In use, the extracorporeal blood circuit is connected to a patient, generally an arm of a patient, and the pump is actuated to withdraw blood from the patient and pump it through the circuit. The whole blood from the patient enters the separator 7 and is separated under pressure into the two fractions. The plasma fraction is pumped from the second chamber 10b to the reaction chamber 8 where the blood percolates through the functionalized cassette bed 17. In the reaction chamber, mediator in the plasma binds to the protease enzyme that is immobilized to the support material, and is cleaved into an inactive form that is released back into the plasma leaving the immobilized enzyme free for another reaction. As a result of the plasma passing through the reaction chamber, the concentration of functional mediator in the plasma is significantly reduced. The thus treated plasma is then pumped to the mixing chamber 15 where it rejoins with the cell rich fraction to form whole blood that is significantly depleted of active mediator protein. The whole blood is returned to the patient via the return line 5.

It will be appreciated that the use of a separator to filter the blood prior to treatment is optional, and that the treatment of whole blood in the reaction chamber forms part of the invention.

### EXPERIMENTAL

### Materials and Methods

### C5a Peptidase Activity Assays

Recombinant C5a was produced as an N-term His-tagged fusion (HT-C5a) in accordance with the method of Toth et al., and chemoattractant activity was verified in an under-agarose migration assay (data not shown). The C5a-ase activity of ScpA was demonstrated in reactions consisting of 42 nM ScpA with 37 μM HT-C5a, in 50 mM Tris/HCl (pH 7.5), 100 mM NaCl, and 5 mM CaCl₂ for 30 min at 20° C. The observed C5a-ase activity was independent of the presence of Complete Mini EDTAfree inhibitor cocktail (Roche). Matrix-assisted laser desorption ionization time-of-flight mass spectrometry analysis of cleaved HT-C5a was performed.

### Results

The activity assay showed that the ScpA cleaved C5a at a single site (FIG. 2a). MS analysis indicated a loss of 830 Da, consistent with the removal of seven residues from the C terminal (FIG. 2b) which removes chemoattractant capabilities.

The invention is not limited to the embodiments hereinbefore described which may be varied in construction and detail without departing from the spirit of the invention.

### REFERENCES

Brown CK, Gu ZY, Matsuka YV, Purushothaman SS, Winter LA, Cleary PP, Olmsted SB, Ohlendorf DH, Earhart CA. Structure of the streptococcal cell wall C5a peptidase. Proc Natl Acad Sci U S A. 2005 Dec. 20; 102(51):18391-6. Epub 2005 Dec. 12. PubMed PMID: 16344483; PubMed Central PMCID: PMC1317908.

Fritzer A, Noiges B, Schweiger D, Rek A, Kungl AJ, von Gabain A, Nagy E, Meinke AL. Chemokine degradation by the Group A streptococcal serine proteinase ScpC can be reconstituted in vitro and requires two separate domains. Biochem J. 2009 Aug. 27; 422(3):533-42. doi: 10.1042/BJ20090278. PubMed PMID: 19552626.

Kaur SJ, Nerlich A, Bergmann S, Rohde M, Fulde M, Zähner D, Hanski E, Zinkernagel A, Nizet V, Chhatwal GS, Talay SR. The CXC chemokine-degrading protease SpyCep of Streptococcus pyogenes promotes its uptake into endothelial cells. J Biol Chem. 2010 Sep. 3; 285(36):27798-805. doi: 10.1074/jbc.M109.098053. Epub 2010 Jun. 18. PubMed PMID: 20562101; PubMed Central PMCID: PMC2934647.

Zinkernagel AS, Timmer AM, Pence MA, Locke JB, Buchanan JT, Turner CE, Mishalian I, Sriskandan S, Hanski E, Nizet V. The IL-8 protease SpyCEP/ScpC of group A Streptococcus promotes resistance to neutrophil killing. Cell Host Microbe. 2008 Aug. 14; 4(2):170-8. doi: 10.1016/j.chom.2008.07.002. PubMed PMID: 18692776; PubMed Central PMCID: PMC2631432.

Sjölinder H, Lövkvist L, Plant L, Eriksson J, Aro H, Jones A, Jonsson AB. The ScpC protease of Streptococcus pyogenes affects the outcome of sepsis in a murine model. Infect Immun. 2008 September; 76(9):3959-66. doi: 10.1128/IAI.00128-08. Epub 2008 Jun. 23. PubMed PMID: 18573900; PubMed Central PMCID: PMC2519448.

Hidalgo-Grass C, Mishalian I, Dan-Goor M, Belotserkovsky I, Eran Y, Nizet V, Peled A, Hanski E. A streptococcal protease that degrades CXC chemokines and impairs bacterial clearance from infected tissues. EMBO J. 2006 Oct. 4; 25(19):4628-37. Epub 2006 Sep. 14. PubMed PMID: 16977314; PubMed Central PMCID: PMC1589981.

Matheson NR, Potempa J, Travis J. Interaction of a novel form of Pseudomonas aeruginosa alkaline protease (aeruginolysin) with interleukin-6 and interleukin-8. Biol Chem. 2006 July; 387(7):911-5. PubMed PMID: 16913841.

J.M. Stewart and J.D. Young, Solid Phase Peptide Synthesis, 2nd edition, Pierce Chemical Company, Rockford, Illinois (1984), in M. Bodanzsky and A. Bodanzsky, The Practice of Peptide Synthesis, Springer Verlag, New York (1984).

Toth, M.J., Huwyler, L., Boyar, W.C., Braunwalder, A.F., Yarwood, D., Hadala, J., Haston, W.O., Sills, M.A., Seligmann, B., Galakatos, N. The pharmacophore of the human C5a anaphylatoxin. Protein Sci. 3:1159-1168, 1994.

1. A method for the extracorporeal treatment of blood, the method comprising removing blood or a blood fraction from a patient and reacting the blood or blood fraction with a protease enzyme that is specific for and capable of irreversibly cleaving functional C5a, thereby reducing an abundance of functional C5a and increasing an abundance of non-functional C5a in the blood or blood fraction compared to that of untreated blood or blood fraction, and returning the blood or the blood fraction to the patient, wherein the protease enzyme is immobilised to a support.

2. The method of claim 1, wherein the protease enzyme is a recombinant bacterial C5a protease comprising SEQ ID NO. 3, SEQ ID NO: 4 or SEQ ID NO: 5.

3. (canceled)

4. The method of claim 1, wherein the reacting step is carried out with an apparatus comprising:
an extracorporeal blood circuit;
a pump configured to provide fluid displacement with the extracorporeal blood circuit; and
a reaction chamber connected to the extracorporeal blood circuit, the reaction chamber configured to receive the blood or blood fraction from the extracorporeal blood circuit and to treat the blood or blood fraction,
wherein the reaction chamber contains the protease enzyme immobilised to the support.

5. The method of claim 4, wherein the protease enzyme is a recombinant bacterial C5a protease comprising SEQ ID NO. 3, SEQ ID NO: 4 or SEQ ID NO: 5.

6. (canceled)

7. The method of claim 4, wherein the apparatus further comprises separating means adapted to separate the blood or blood fraction into a C5a-containing fraction and a non-C5a containing fraction, wherein the reaction chamber receives the C5a-containing fraction.

8. The method of claim 7, wherein the protease enzyme is a recombinant bacterial C5a protease comprising SEQ ID NO. 3, SEQ ID NO: 4 or SEQ ID NO: 5.

9. (canceled)

10. The method of claim 7, wherein the apparatus further comprises means configured to recombine the treated C5a-containing fraction with the non-C5a containing fraction.

11. The method of claim 1, wherein the support includes a coordinated transition metal ion and one or more functional groups; the C-terminus of the protease enzyme comprises a first tag and a second tag, the second tag being separated from the first tag by a spacer; the first tag comprises a motif capable of covalently reacting with the one or more functional groups; and the second tag comprises a motif capable of interacting with the coordinated transition metal ion.

12. The method of claim 11, wherein the first tag is poly-lysine, poly-glutamate, or poly-cysteine, and the second tag is poly-histidine.

13. The method of claim 12, wherein the protease enzyme is a recombinant bacterial C5a protease comprising SEQ ID NO. 3, SEQ ID NO: 4, or SEQ ID NO: 5.

14. The method of claim 1, wherein the support includes a silica material, a methacrylate, a polyacrylamide, a polypyrrole, or a polysaccharide.

15. The method of claim 14, wherein the silica material is selected from the group consisting of mesoporous silica, a monodispersed mesoporous silicate, and a Ni2+-modified mesoporous silica.

16. The method of claim 1, wherein the support comprises a multiplicity of beads and the protease enzyme is irreversibly immobilized to a surface of the beads.

17. A method for treating sepsis, the method comprising removing blood or a blood fraction from a patient having sepsis and reacting the blood or blood fraction with a protease enzyme that is specific for and capable of irreversibly cleaving functional C5a, thereby reducing an abundance of functional C5a in the blood or blood fraction, and returning the blood or the blood fraction to the patient, wherein the protease enzyme is immobilised to a support.

18. The method of claim 17, wherein the protease enzyme is a recombinant bacterial C5a protease comprising SEQ ID NO. 3, SEQ ID NO: 4, or SEQ ID NO: 5.

19. (canceled)

20. (canceled)


SEQUENCE LISTING (6 sequences)

SEQ ID NO: 1 - DNA, 3015 bp, Streptococcus pyogenes
  gene 1..3015: note = Gene encoding ScpA
  source 1..3015: mol_type = genomic DNA
ggatccaatactgtgacagaagacactcctgctaccgaacaagccgtagaaaccccacaaccaacagcggtttctgaggaagcaccatcatcatcaaaggaaaccaaaatcccacaaactcctggtgatgcagaagaaacagtagcagatgacgctaatgatctagcccctcaagctcctgctaaaactgctgatacaccagcaacctcaaaagcgactattagggatttgaacgacccttctcaggtcaaaaccctgcaggaaaaagcaggcaagggagctgggactgttgttgcagtgattgatgctggttttgataaaaatcatgaagcgtggcgcttaacagacaaaactaaagcacgttaccaatcaaaagaagatcttgaaaaagctaaaaaagagcacggtattacctatggcgagtgggtcaatgataaggttgcttattaccacgattatagtaaagatggtaaaaccgctgtcgatcaagagcacggcacacacgtgtcagggatcttgtcaggaaatgctccatctgaaacgaaagaaccttaccgcctagaaggtgcgatgcctgaggctcaattgcttttgatgcgtgtcgaaattgtaaatggactagcagactatgctcgtaactacgctcaagctatcagagatgctgtcaacttgggagctaaggtgattaatatgagctttggtaatgctgcactagcttacgccaaccttccagacgaaaccaaaaaagcctttgactatgccaaatcaaaaggtgttagcattgtgacctcagctggtaatgatagtagctttgggggcaaaacccgtctacctctagcagatcatcctgattatggggtggttgggacgcctgcagcggcagactcaacattgacagttgcttcttacagcccagataaacagctcactgaaactgctacggtcaaaacagacgatcatcaagctaaagaaatgcctgttctttcaacaaaccgttttgagccaaacaaggcttacgactatgcttatgctaatcgtgggatgaaagaagatgattttaaggatgtcaaaggcaaaattgcccttattgaacgtggtgatattgatttcaaagataagattgcaaacgctaaaaaagctggtgctgtaggggtcttgatctatgacaatcaagacaagggcttcccgattgaattgccaaatgttgatcagatgcctgcggcctttatcagtcgaaaagacggtctcttattaaaagacaattctaaaaaaaccatcaccttcaatgcgacacctaaggtattgccaacagcaagtgacaccaaactaagccgcttctcaagctggggtttgacagctgacggcaatattaagccagatattgcagcacccggccaagatattttgtcatcagtggctaacaacaagtatgccaaactttctggaactagtatgtctgcgccattggtagcgggtatcatgggactattgcaaaagcaatatgagacacagtatcctgatatgacaccatcagagcgtcttgatttagctaaaaaagtattgatgagctcagcaactgccttatatgatgaagatgaaaaagcttatttttctcctcgccaacaaggagcaggagcagtcgatgctaaaaaagcttcagcagcaacgatgtatgtgacagataaggacaatacctcaagcaaggttcacctgaacaatgtttctgataaatttgaagtaacagtaacagttcacaacaaatctgataaacctcaagagttgtattaccaagcaactgttcaaacagataaagtagatggaaaacactttgccttggctcctaaagcattgtatgagacatcatggcaaaaaatcacaattccagccaatagcagcaaacaagtcaccgttccaatcgatgctagtcgatttagcaaggacttgcttgcccaaatgaaaaatggctatttcttagaaggttttgttcgtttcaaacaagatcctaaaaaagaagagcttatgagcattccatatattggtttccgaggtgattttggcaatctgtcagccttagaaaaaccaatctatgatagcaaagacggtagcagctactatcatgaagcaaatagtgatgccaaagaccaattagatggtgatggattacagttttacgctctgaaaaataactttacagcacttaccacagagtctaacccatggacgattattaaagctgtcaaagaaggggttgaaaacatagaggatatcgaatcttcagagatcacagaaaccatttttgcaggtacttttgcaaaacaagacgatgatagccactactatatccaccgtcacgctaatggcaaaccatatgctgcgatctctccaaatggggacggtaacagagattatgtccaattccaaggtactttcttgcgtaatgctaaaaaccttgtggctgaagtcttggacaaagaaggaaatgttgtttggacaagtgaggtaaccgagcaagttgttaaaaactacaacaatgacttggcaagcacacttggttcaacccgttttgaaaaaacgcgttgggacggtaaagataaagacggcaaagttgttgttaacggaacctacacctatcgtgtccgctacactccgattagctcaggtgcaaaagaacaacacactgattttgatgtgattgtagacaatacgacacctgaagtcgcaacatcggcaacattctcaacagaagatcgtcgtttgacacttgcatctaaaccaaaaaccagccaaccgatttaccgtgagcgtattgcttacacttatatggatgaggatctgccaacaacagagtatatttctccaaatgaagatggtacctttactcttcctgaagaggctgaaacaatggaaggcggtactgttccattgaaaatgtcagactttacttatgttgttgaagatatggctggtaacatcacttatacaccagtgactaagctattggagggccactcttaa

SEQ ID NO: 2 - protein, 1007 aa, Streptococcus pyogenes
  REGION 1..1007: note = ScpA pro-protease
GPLGSNTVTEDTPATEQAVETPQPTAVSEEAPSSSKETKIPQTPGDAEETVADDANDLAPQAPAKTADTPATSKATIRDLNDPSQVKTLQEKASKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTDDHQAKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNSKKTITFNATPKVLPTASDTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVVNGTYTYRVRYTPISSGAKEQHTDFDVIVDNTTPEVATSATFSTEDRRLTLASKPKTSQPIYRERIAYTYMDEDLPTTEYISPNEDGTFTLPEEAETMEGGTVPLKMSDFTYVVEDMAGNITYTPVTKLLEGHS

SEQ ID NO: 3 - protein, 961 aa, Streptococcus pyogenes
  REGION 1..961: note = ScpA mature protease
AEETVADDANDLAPQAPAKTADTPATSKATIRDLNDPSQVKTLQEKASKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTDDHQAKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNSKKTITFNATPKVLPTASDTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVVNGTYTYRVRYTPISSGAKEQHTDFDVIVDNTTPEVATSATFSTEDRRLTLASKPKTSQPIYRERIAYTYMDEDLPTTEYISPNEDGTFTLPEEAETMEGGTVPLKMSDFTYVVEDMAGNITYTPVTKLLEGHS

SEQ ID NO: 4 - protein, 954 aa, Streptococcus pyogenes
  REGION 1..954: note = ScpA protease variant
DANDLAPQAPAKTADTPATSKATIRDLNDPSQVKTLQEKASKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTDDHQAKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNSKKTITFNATPKVLPTASDTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVVNGTYTYRVRYTPISSGAKEQHTDFDVIVDNTTPEVATSATFSTEDRRLTLASKPKTSQPIYRERIAYTYMDEDLPTTEYISPNEDGTFTLPEEAETMEGGTVPLKMSDFTYVVEDMAGNITYTPVTKLLEGHS

SEQ ID NO: 5 - protein, 943 aa, Streptococcus pyogenes
  REGION 1..943: note = ScpA protease variant
KTADTPATSKATIRDLNDPSQVKTLQEKASKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTDDHQAKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNSKKTITFNATPKVLPTASDTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVVNGTYTYRVRYTPISSGAKEQHTDFDVIVDNTTPEVATSATFSTEDRRLTLASKPKTSQPIYRERIAYTYMDEDLPTTEYISPNEDGTFTLPEEAETMEGGTVPLKMSDFTYVVEDMAGNITYTPVTKLLEGHS

SEQ ID NO: 6 - protein, 74 aa, Homo sapiens
  REGION 1..74: note = Human C5a protein
MLQKKIEEIAAKYKHSVVKKCCYDGACVNNDETCEQRAARISLGPRCIKAFTECCVVASQLRANISHKDMQLGR